(19) United States
(12) Patent Application Publication
(10) Pub. No.: US 2008/0312641 A1
(43) Pub. Date: Dec. 18, 2008

(54) LASER TREATMENT OF VASCULAR LESIONS, PIGMENTED LESIONS AND HAIR WITH A PULSED-DYE LASER

(76) Inventor: Eric F. Bernstein, Gladwyne, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/137,626

(22) Filed: Jun. 12, 2008

Related U.S. Application Data
(60) Provisional application No. 60/929,141, filed on Jun. 14, 2007.

(51) Int. Cl. A61B 18/20

(52) U.S. Cl. 606/9

(57) ABSTRACT

There is disclosed a method of treating one or more skin conditions using pulsed, scanned, or continuously administered laser light in multiple wavelength ranges. The skin conditions that can be treated include vascular lesions and/or dermal remodeling by administering to the skin, energy having a wavelength ranging from 570-598 nanometers, and reducing or removing unwanted hair by administering to the skin energy having a wavelength ranging from 550-560 nanometers or from 598-1100 nanometers. The various energies are provided by the same laser and can be administered in any order or simultaneously.

This application claims the benefit of domestic priority to U.S. Provisional Application 60/929,141, filed Jun. 14, 2007, which is herein incorporated by reference in its entirety.

The present disclosure relates to a method of treating one or more skin conditions using a single laser source that produces multiple wavelength ranges. The various energies can be administered in any order or simultaneously.

The traits associated with skin aging are largely due to chronic sun exposure. Five main changes occur in photodamaged skin: fine lines and wrinkles, enlarged pores, spider veins, sagging skin, and brown spots such as solar lentigos and ephiledes (freckles). The pulsed-dye laser has been the mainstay of removing unwanted blood vessels such as those that occur in port-wine stain birthmarks, in sun-damaged skin, in those with rosacea, and in scars for example since the 1980s.

The 577-600 nm laser light typically made available from the pulsed-dye laser is also absorbed by melanin pigment. The strong absorption characteristics in hemoglobin competing for this wavelength has made these lasers less than optimal for treating pigmented lesions such as ephiledes and solar lentigos. The pulsed-dye laser has been shown to improve wrinkles in sun-damaged skin presumably due to the inflammation that occurs immediately following treatment.

In addition to pulsed dye lasers, there are devices that use filtered intense pulsed light (IPL's) that contains many wavelengths of light. The advantage of these systems is that they are inexpensive to produce, and can be used to treat a range of conditions in the skin with less specificity. However, intense pulsed light devices that emit broad spectrum light are not optimal for treating blood vessels since much of the emitted light is not absorbed by hemoglobin and thus contributes to non-specific heating of the skin. This and other factors associated with IPL's present a potential of greater side-effects than pulsed dye lasers. Melanin, unlike hemoglobin, has a very large range of absorbing wavelengths, and can be targeted by intense pulsed light devices fairly effectively. It is known that melanin absorbs the shorter wavelengths best, since these are the most damaging to our body and melanin has presumably evolved as a protective mechanism against solar radiation. Therefore, ultraviolet wavelengths from 290-400 nm are absorbed the strongest, with decreasing absorption at longer wavelengths, absorbing into the infrared.

Hair removal is one of the functions attributed to the intense pulsed light (IPL) devices that are sold as multi-purpose laser-like devices used for facial rejuvenation, hair removal, and vein treatment. However, because IPL devices emit a broad spectrum of light and not a single or few wavelengths, they are often less predictable in their effects as compared to a laser. Having true laser with multi-functionality would be a significant improvement over IPL devices.

To make the pulsed-dye laser an optimal, single device for treating a myriad of conditions, it needs to have multiple functions. For example, currently, pulsed-dye lasers are capable of delivering wavelengths longer than 600 nm which would make it good for reducing hair growth via an inflammatory mechanism that stimulates a prolonged telogen phase.

Pulsed-dye lasers are capable of emitting a variety of wavelengths of light depending upon the dye used and the use of diffraction gradients or prisms to select various wavelengths from the spectrum of light emitted by exciting the laser dye. To make treatment unwanted hair possible with the pulsed-dye laser, there is described a dye laser capable of emitting wavelengths away from the peak absorption of hemoglobin and long enough and with a large enough spot size to allow for deeper penetration of skin for the specific purpose of reducing hair growth. Thus, there is disclosed a method to reduce or remove unwanted hair with a pulsed dye laser. This enables one to treat a variety of skin conditions using a single source pulsed-dye laser.

### SUMMARY OF THE INVENTION

There is disclosed a method of treating one or more skin conditions using pulsed, scanned, or continuously administered laser light in multiple wavelength ranges. In one embodiment, the method comprises administering to the skin, from a single dye laser:

(a) energy having a wavelength ranging from 570-598 nanometers; and

(b) energy having a wavelength ranging from 550-560 nanometers or from 598-1100 nanometers, wherein the energy in (a) is administered prior, subsequent or simultaneous to the energy in (b).

In one embodiment, the energy in (a) ranges from 590 to 598 nanometers and is administered for a time sufficient to reduce or remove vascular lesions and/or for dermal remodeling. In addition, the energy in (b) may range from 555 to 565 or from 600 to 610 nanometers is administered for a time sufficient to reduce or remove unwanted hair.

There is also disclosed a method of administering to the skin at least two wavelengths of light from the same source. This method may comprise sequentially administering a (a) first wavelength of light sufficient for treating unwanted vasculature, such as from 570-598 nanometers, and (b) a second wavelength outside of the absorption peaks for hemoglobin and sufficient for treating unwanted hair, such as from 550-560 nanometers or from 598-1100 nanometers.

### DETAILED DESCRIPTION OF THE INVENTION

There is disclosed a dye laser with tunable wavelengths for the treatment of multiple skin conditions, including unwanted hair. In this embodiment the skin is irradiated with light (electromagnetic radiation) having a wavelength and spot size sufficient to induce an inflammatory and/or urticarial response in skin thereby reducing terminal hair in skin. Using a dye laser to administer wavelengths outside of the absorption peaks for hemoglobin typically used to treat unwanted blood vessels enables hair removal without causing bruising (purpura) in the skin.

It is known that hair can be removed using optical pulses, such as by using light energy to destroy hair follicles in the desired region. Parameters that can be varied to effect complete damage of desired portions of the hair follicles in the region with minimal damage to surrounding tissue and to the patient's epidermis are described in U.S. Pat. Nos. 5,735,844 and 5,595,568, which are herein incorporated by reference.

Pulsed-dye lasers typically use wavelengths of 585 or 595 nanometers to treat blood vessels. While these wavelengths are optimal for removing blood vessels, they are not optimal for hair removal due to the strong hemoglobin absorption limiting penetration of the laser light to a superficial depth, not sufficiently deep to induce hair reduction. This type of treatment usually incorporates some form of epidermal cooling and is administered to the entire face or entire cosmetic unit (cheek, chin, nose for example) to be treated.

By treating the entire face with a wavelength not as highly absorbed by hemoglobin outside the wavelengths absorbed by hemoglobin, 574-598 nanometers, melanin can be targeted while avoiding strong blood absorption. This has the advantage of reducing the likelihood of purpura following treatment, and enabling treatment of the entire skin surface for hair reduction or removal. This has the advantage of adding functionality to the pulsed-dye laser, enabling the added function of hair removal to this device.

Pulsed-dye lasers have been developed in the past that were capable of being tuned for emission of wavelengths ranging from 585 nanometers to 600 nanometers in 5 nanometer increments. However, wavelengths longer than 595 nanometers were not often used, since they were relatively ineffective at treating vascular lesions. Current lasers do not generally offer this broad range of wavelengths.

Development of a pulsed-dye laser capable of delivering vascular-specific wavelengths in the 574-598 nanometer range, as well as wavelengths outside of that range below 574 nanometers or above 600 nanometers for the purpose of treating unwanted hair in the skin would dramatically increase the utility of pulsed-dye lasers. A diffraction gradient, prism, or other means of altering the delivered wavelength emitting from a dye laser would enable the delivery of treatments to reduce unwanted blood vessels, unwanted pigmentation, induce skin remodeling and enable reduction or removal of unwanted hair.

These different wavelengths could be delivered singly, sequentially, or simultaneously to achieve the desired outcome of removing unwanted pigmentation, unwanted vasculature and unwanted hair. A dye laser could be continuous and swept over the skin to result in the effect of a pulse, or pulsed as most lasers in clinical use today are.

A laser capable of delivering at least two wavelengths, at least one for hemoglobin, and at least one targeting melanin pigment and not at the peak absorption range for hemoglobin, would enable more complete treatment of sun-damaged skin and unwanted hair. This system would have significant advantages over intense pulsed light (IPL) systems because there would be narrow ranges of wavelengths being administered as opposed to the broad wavelength ranges delivered with intense pulsed light devices.

The administration of more discrete wavelengths permits more accurate prediction of laser effects than is possible when using broad spectrum light sources such as intense pulsed light devices. Intense pulsed light devices emit a broad range of wavelengths, making prediction of clinical outcomes more difficult. Using laser energy of specific wavelengths permits a more accurate estimation of the proper energy to be used for a given patient, enhancing effectiveness and limiting side effects.

Pulsed-dye lasers are currently used mostly for treating vascular lesions such as port-wine stain birthmarks, rosacea, facial veins and diffuse redness, scars and lower extremity spider veins. Pulsed-dye lasers are capable of emitting a variety of wavelengths depending upon the type of dye used in the laser and also through the use of diffraction gradients or prisms within the laser. Thus, such lasers can be tuned to different wavelengths.

Incorporating the ability to tune to wavelengths not in the peak absorption range of hemoglobin would enable the treatment of unwanted hair. Melanin pigment absorbs light over a much broader range than hemoglobin. Non-limiting examples for melanin targeting and possible hair removal would be from approximately 550-560 nanometers or 605-1100 nanometers. The desired wavelength would depend upon how much energy could be delivered at the various wavelengths.

Using a single dye in the dye laser to achieve both vascular-specific wavelengths and those outside the range of hemoglobin for treating pigmented lesions and unwanted hair would simplify the laser and lower cost, as compared to having a laser with multiple dye chambers, although multiple dye changers is a possible design that would enable delivery of multiple wavelengths. Thus wavelengths up to approximately 615 nm would be most easily achievable while also delivering vascular-specific wavelengths such as 595 nm.

Combining the ability to deliver a wavelength of light not in the peak absorption range of hemoglobin would enable treatment of epidermal and dermal melanocytic lesions and unwanted hair without affecting dermal blood vessels and causing a bruise. The wavelength specific for hemoglobin, most commonly 595 nanometers in current clinical practice, could be administered before, during or concurrently with the 595 nanometer wavelength. This would enable treatment of melanocytic lesions such as ephiledes or lentigos with a lower risk of purpura.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and in the attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result. The percentages are given on a weight basis.

What is claimed is:

1. A method of treating one or more skin conditions using pulsed, scanned, or continuously administered laser light in multiple wavelength ranges, said method comprising administering to the skin, from a single dye laser:
a) energy having a wavelength ranging from 570-598 nanometers; and
b) energy having a wavelength ranging from 550-560 nanometers or from 598-1100 nanometers, wherein the energy in (a) is administered prior, subsequent or simultaneous to the energy in (b).

2. The method of claim 1, wherein the energy in (a) is administered for a time sufficient to reduce or remove vascular lesions and/or for dermal remodeling.

3. The method of claim 1, wherein the energy in (a) is administered for a time sufficient to reduce or remove unwanted hair.

4. The method of claim 1, wherein the energy administered in (a) ranges from 590 to 598 nanometers and the energy administered in (b) ranges from 555 to 565 or from 600 to 610 nanometers.

5. A method of administering to the skin at least two wavelengths of light from a single dye laser, said method comprising sequentially administering a (a) first wavelength of light sufficient for treating unwanted vasculature, and (b) a second wavelength outside of the absorption peaks for hemoglobin and sufficient for treating unwanted hair.

6. The method of claim 5, wherein said first wavelength (a) ranges from 570-598 nanometers.

7. The method of claim 5, wherein said second wavelength (b) ranges from 550-560 nanometers or from 598-1100 nanometers.
